(19) United States
(12) Patent Application Publication
(10) Pub. No.: US 2007/0233165 A1
(43) Pub. Date: Oct. 4, 2007

(54) Tongue cleaning device

(76) Inventor: Robert K. Wagstaff, Springville, UT (US)

Correspondence Address: DANIEL P. MCCARTHY, P.O. BOX 71550, SALT LAKE CITY, UT, 84171-0550, US

(21) Appl. No.: 11/397,312

(22) Filed: Apr. 4, 2006

(51) Int. Cl. A61B 17/24

(52) U.S. Cl. 606/161; 15/111

(57) ABSTRACT

Devices for cleaning the human tongue and methods for making such devices are disclosed. The tongue cleaning device may include a tongue scraper and a tongue scraper guide. The guide may be a in a fixed position or it may be a movable drum roller guide. The surface of the scraper guide may be smooth or it may have protrusions, indentations or bristles. The tongue cleaning device may also consist of a tongue scraper, tongue scraper guide, and tongue brush.

### BACKGROUND OF THE INVENTION

This invention relates to the field of devices for cleaning the human tongue, and methods for making the same. During the past few years several tongue cleaning devices have been patented to clean the tongue. These devices have been devised because the general public has become more aware of the importance of cleaning the tongue in order to control bad breath (halitosis) and to maintain good oral health. The early tongue cleaning devices were simple in design and several are now commercially available. This invention identifies improvements in the devices to make them more effective and safer to use.

The tongue surface contains many small openings where taste buds and salivary glands reside which result in the tongue surface being uneven. These small openings can contain waste material that adversely affects the function of the salivary glands and taste buds. These areas harbor microorganisms that colonize and produce odors and deposit waste materials. In order to remove this waste material which is composed of organic matter various cleaning devices have been invented which consist of a tongue scraper or brush. These scrapers and brushes can damage the soft tissue of the tongue because of the pressure that is exerted by the user to force the scraper or brush to reach down into these pockets and pores.

The object of the inventions is to provide devices for cleaning the human tongue, and methods for making devices for cleaning the human tongue. The device includes a tongue scraper and a scraper guide. This guide causes the surface of the tongue to be depressed and then be released in such a way as to present the surface of the tongue to the scraper as the tongue surface is coming back to normal. As this depression in the tongue surface comes back to normal it modifies the surface openings and permits the removal of the waste material without excessive pressure or injury to the soft surface tissue of the tongue. The scraper guide may be solid or movable in nature. A third device includes a scraper, a guide, and a brush in combination.

### BRIEF DESCRIPTION OF THE DRAWINGS

FIG. 1 depicts a longitudinal cross-section side view of the human tongue and surrounding anatomy.

FIG. 2 depicts a latitudinal front view of a tongue cleaner with a rigid guide.

FIG. 3 depicts a longitudinal side view of a tongue cleaner with a rigid guide.

FIG. 4 depicts a longitudinal close up side view of a tongue cleaner with a rigid guide showing how the tongue cleaner affects the cleaning of the tongue surface.

FIG. 5 depicts a longitudinal close up side view of a rigid guide showing a rough surface.

FIG. 6 depicts a latitudinal close up front view of a rigid guide showing a rough surface.

FIG. 7 depicts latitudinal front view of a tongue cleaner with a movable drum roller guide.

FIG. 8 depicts a longitudinal side view of a tongue cleaner with a movable drum roller guide.

FIG. 9 depicts a longitudinal close up side view of the tongue cleaner head with a smooth surface movable drum roller guide.

FIG. 10 depicts a latitudinal close up front view of a movable drum roller guide with a smooth surface.

FIG. 11 depicts a latitudinal close up front view of a movable drum roller guide with a rough surface.

FIG. 12 depicts a latitudinal close up front view of a movable drum roller guide with a surface of pointed bristles.

FIG. 13 depicts a latitudinal front view of a tongue cleaner with a scraper, a movable drum roller guide, and a brush in combination.

FIG. 14 depicts a longitudinal side view of a tongue cleaner with a scraper, a movable drum roller guide, and a brush in combination.

FIG. 15 depicts a longitudinal close up side view of a scraper, a movable drum roller guide, and a brush in combination. The bristles on this brush are individual, pointed, and pliable.

FIG. 16 depicts a latitudinal front view of the method of attaching the movable drum roller guide to the frame of the tongue cleaner head.

FIG. 17 depicts the molding stage of a tongue cleaner with a scraper and a rigid guide device in a one-step injection molding process.

FIG. 18 depicts the ejection stage of a tongue cleaner with a scraper and a rigid guide device in an injection molding process.

FIG. 19 depicts the longitudinal side view close up showing the relationship of the tongue scraper and the movable drum roller guide method of attachment to the device head.

FIG. 20 depicts the latitudinal front view close up showing the two movable drum roller guide attachment points on the device head.

FIG. 21 depicts the molding stage of a one step injection mold for the production of the movable drum roller guide.

FIG. 22 depicts a latitudinal side view of an injection molding machine.

FIG. 23 depicts a latitudinal side view of the injection system of an injection molding machine.

### DETAILED DESCRIPTION

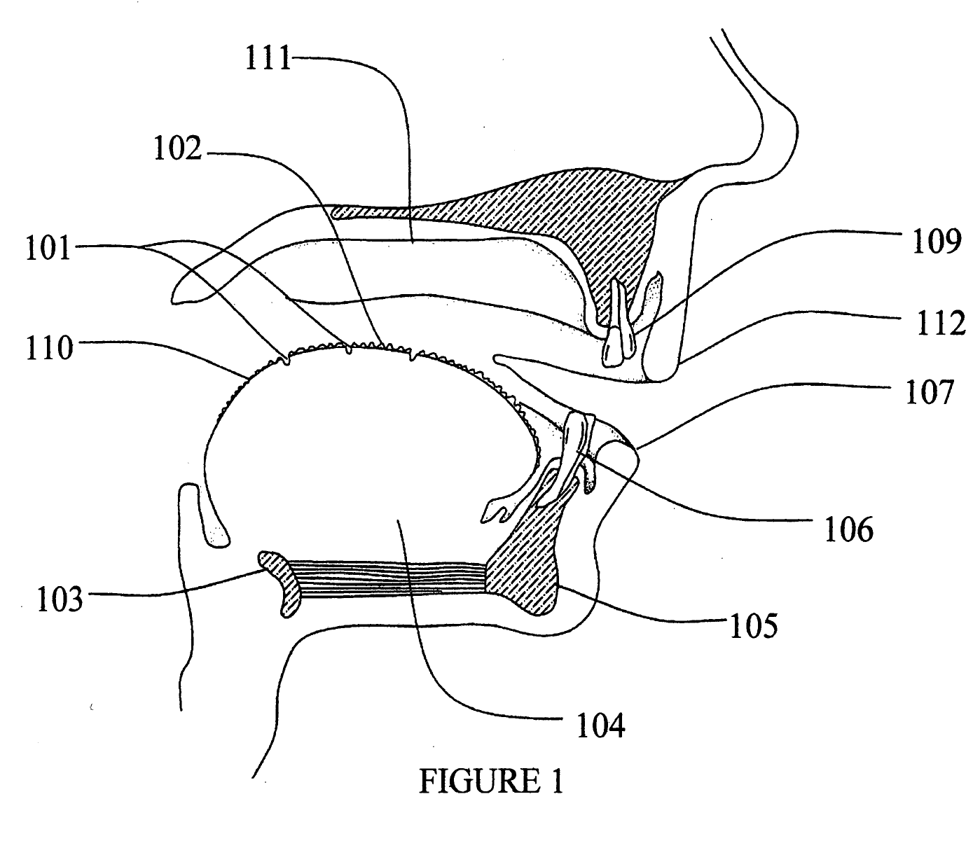

Referring to FIG. 1, the human tongue 110 is substantially all muscle 104, and has a surface made up of taste buds 101 and papillae (also referred to as mucous membrane) 102. The hypoid bone 103 is directly beneath the tongue 110, and the hard palate 111 is directly above the tongue 110. The jawbone 105, lower teeth 106, lower lip 107, upper lip 112 and upper teeth 109 outline the human mouth orifice. This cross-sectional view illustrates in a minor degree the roughness and crevices of the human tongue, which present a particularly difficult cleaning task.

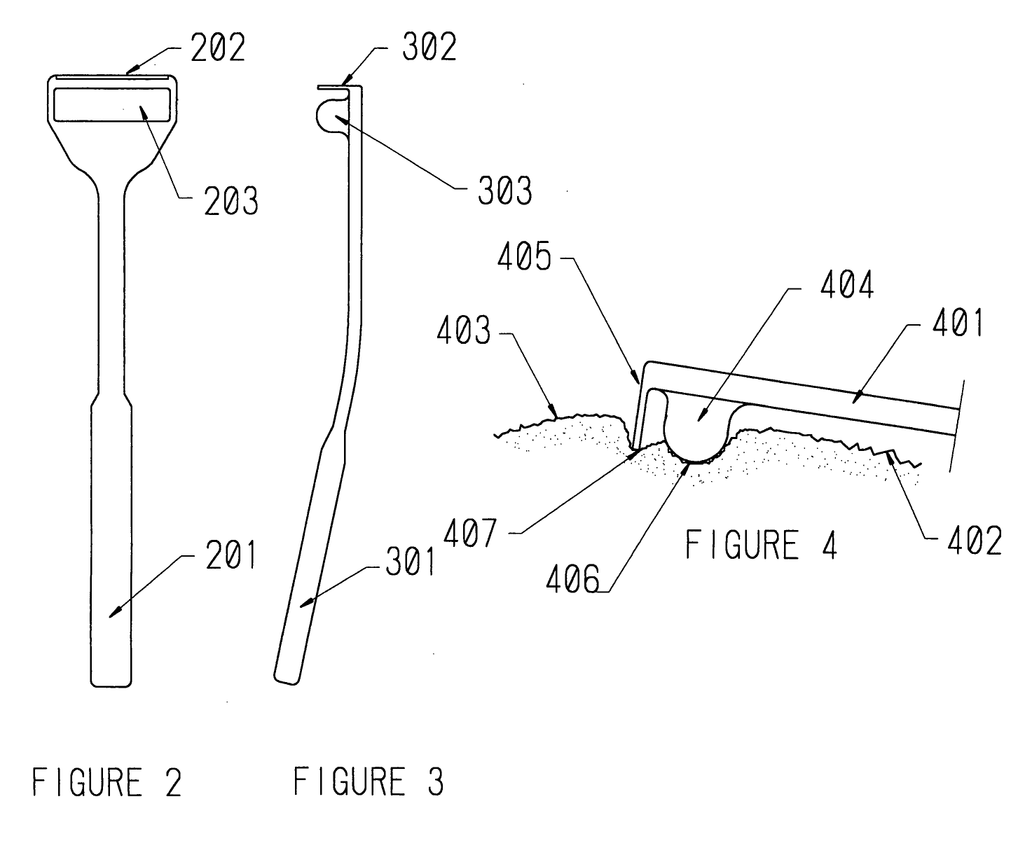

An embodiment of a tongue-cleaning device of the invention is depicted in FIG. 2. The device depicted 201 is a combination tongue scraper 202 and a rigid guide 203. The rigid guide 203 is constructed in such a way as to be elongated in front of the tongue scraper 202. This guide causes a depression in the tongue and takes most of the pressure that is applied to the device during the cleaning process. This reduces the scraping pressure of the tongue scraper 202 and assists in the cleaning process by reducing the chance of injury to the tongue by the tongue scraper 202.

FIG. 3 depicts a latitudinal side view of the tongue cleaning device 301 composed of a tongue scraper 302 and a rigid guide 303. The height of the scraper 302 and the guide 303 are the same so that they both touch the surface of the tongue at the same time.

FIG. 4 depicts the position of the tongue cleaning device 401 on the tongue surface 403 during the cleaning activity. The surface of the tongue 403 consists of many indentations 402 and is uneven in nature. It is a very soft pliable surface that is easily damaged or injured. Note that the top point of the rigid guide 404 causes a depression 406 to the surface of the tongue. As the device 401 is moved across the surface of the tongue 403 the tongue begins to come back to normal 407. At this critical point the scraper 405 meets the tongue surface 403 at an angle conducive to better cleaning. These enables the tongue scraper 405 to more efficiently and effectively remove the waste material from the tongue surface 403 because the tongue surface is presented to the tongue scraper 405 at a different angle than it would normally be presented without the presence of the guide 404.

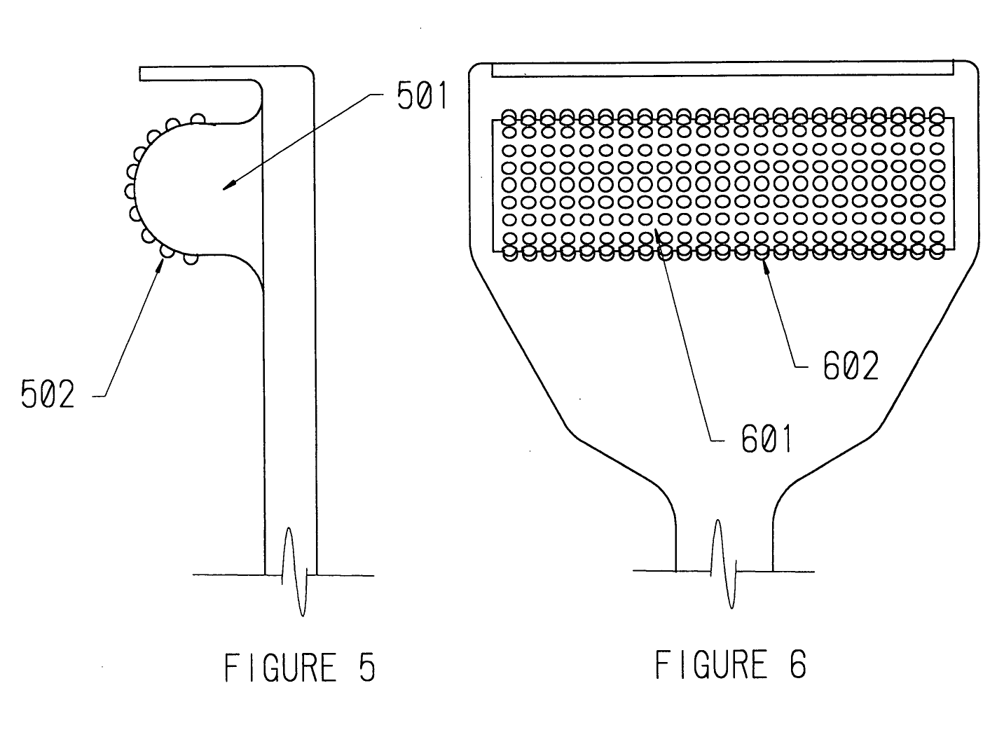

FIG. 5 depicts the latitudinal side view close up of the rigid guide 501 showing that the surface of the guide 502 could be rough in nature rather than smooth in nature.

FIG. 6 depicts the longitudinal front view close up of the rigid guide 601 showing the surface to contain many bumps 602 or protrusions 602. This rough surface causes a “drag” or abrasiveness on the surface of the tongue and helps to break up or dislodge the waste material that is present on the tongue surface.

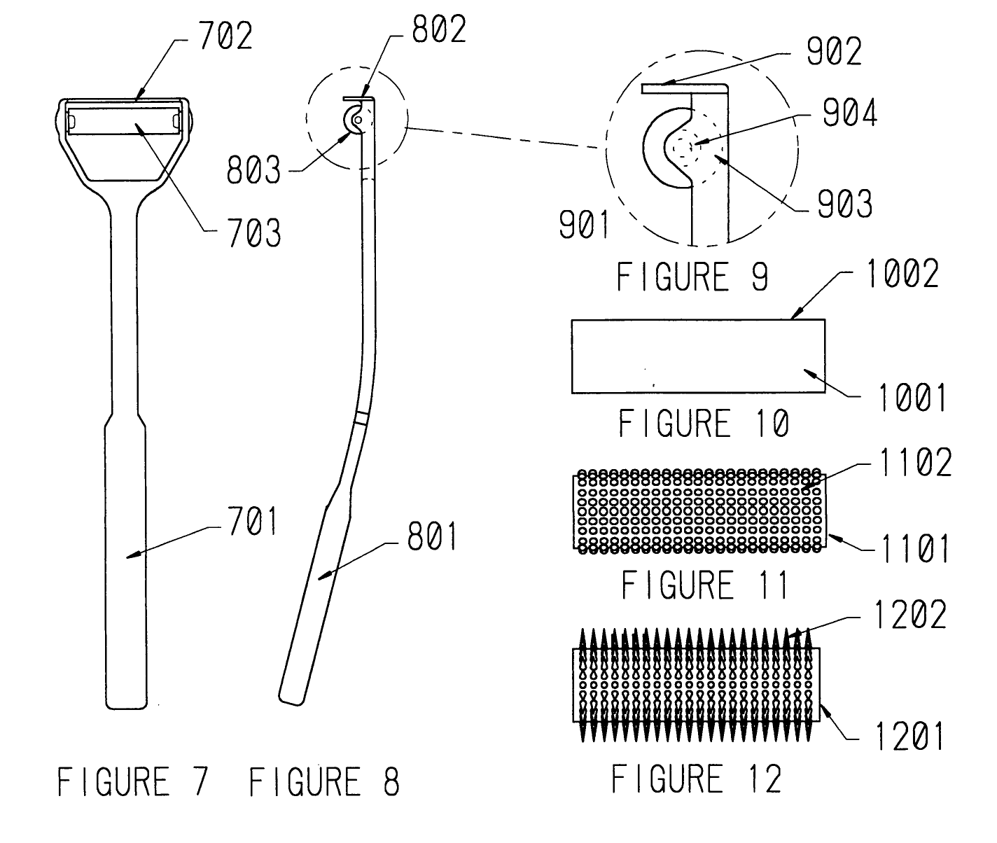

FIG. 7 depicts a longitudinal front view of a tongue cleaning device 701 composed of a tongue scraper 702 and a movable drum roller guide 703.

FIG. 8 depicts a latitudinal side view of a tongue cleaning device 801 composed of a tongue scraper 802 and a movable drum roller guide 803.

FIG. 9 depicts a latitudinal side view of the head of a tongue cleaning device 901 composed of a tongue scraper 902 and a movable drum roller guide 903. This guide rolls along the tongue as the device is being used causing an indentation in the surface of the tongue (reference paragraph 0031). The movable drum roller guide 903 is attached to the tongue cleaning head 901 in such way so that the movable drum roller guide will turn as the head is moved across the tongue surface.

FIG. 10 depicts a longitudinal front view of a movable drum roller guide 1001 showing the surface 1002 to be smooth.

FIG. 11 depicts a longitudinal front view of a movable drum roller guide 1101 showing the surface 1102 to be rough in nature either by bumps or indentations in or on the surface.

FIG. 12 depicts a longitudinal front view of a movable drum roller guide 1201 showing the surface 1202 to contain numerous bristles which are individual and pointed and flexible. These bristles are not a family of bristles but individual bristles and are an integral part of the guide surface. These bristles penetrate the tongue surface and reach into the various indentations that are present on the tongue surface and help to break up and remove the waste material that is present.

FIG. 13 depicts a longitudinal front view of a tongue cleaning device 1301 consisting of a tongue scraper 1302, a movable drum roller guide 1303 and a tongue brush 1304. The bristles of this brush are individual bristles and are randomly placed on the tongue cleaning device head 1305.

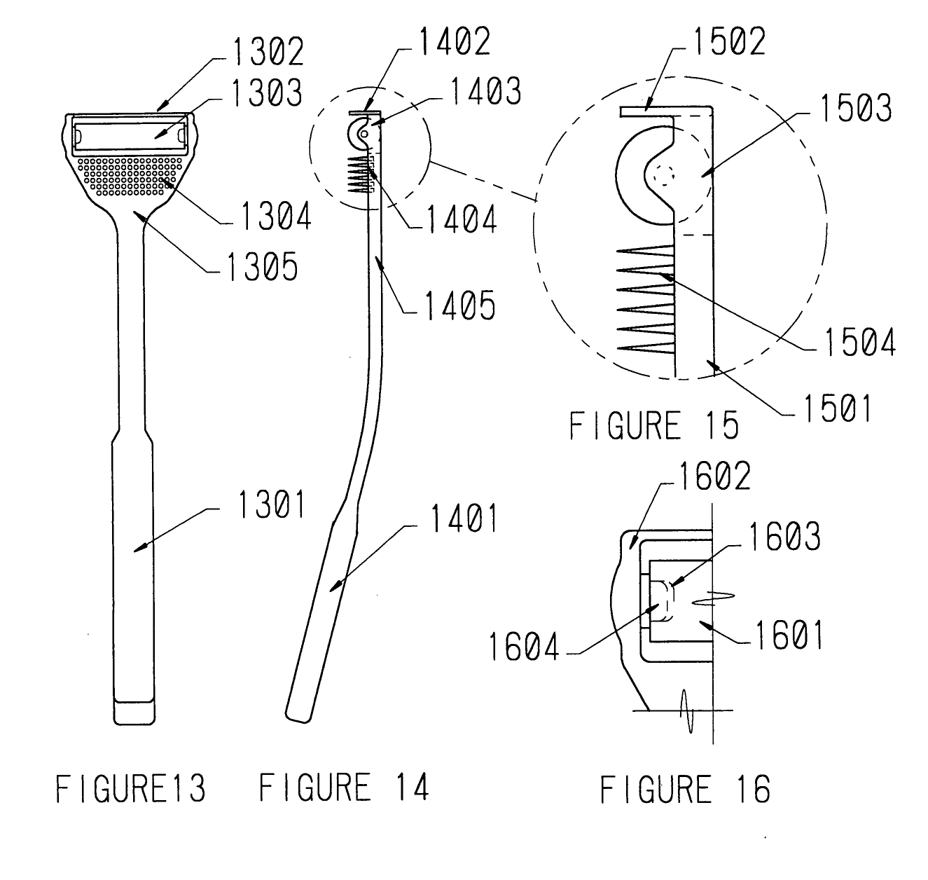

FIG. 14 depicts a latitudinal side view of a tongue cleaning device 1401 consisting of a tongue scraper 1402, a movable drum roller guide 1403 and a tongue brush 1404. The combination of the brush 1404, the guide 1403 and the scraper 1402 results in the tongue being brushed and scraped at the same time. The guide 1403 absorbs the pressure applied by the tongue cleaning device 1401 on the surface of the tongue. This combination is the most complete and efficient cleaning method.

FIG. 15 depicts a latitudinal side view close up of the head of a tongue cleaning device 1501 containing a scraper 1502, a movable drum roller guide 1503 and a tongue brush 1504.

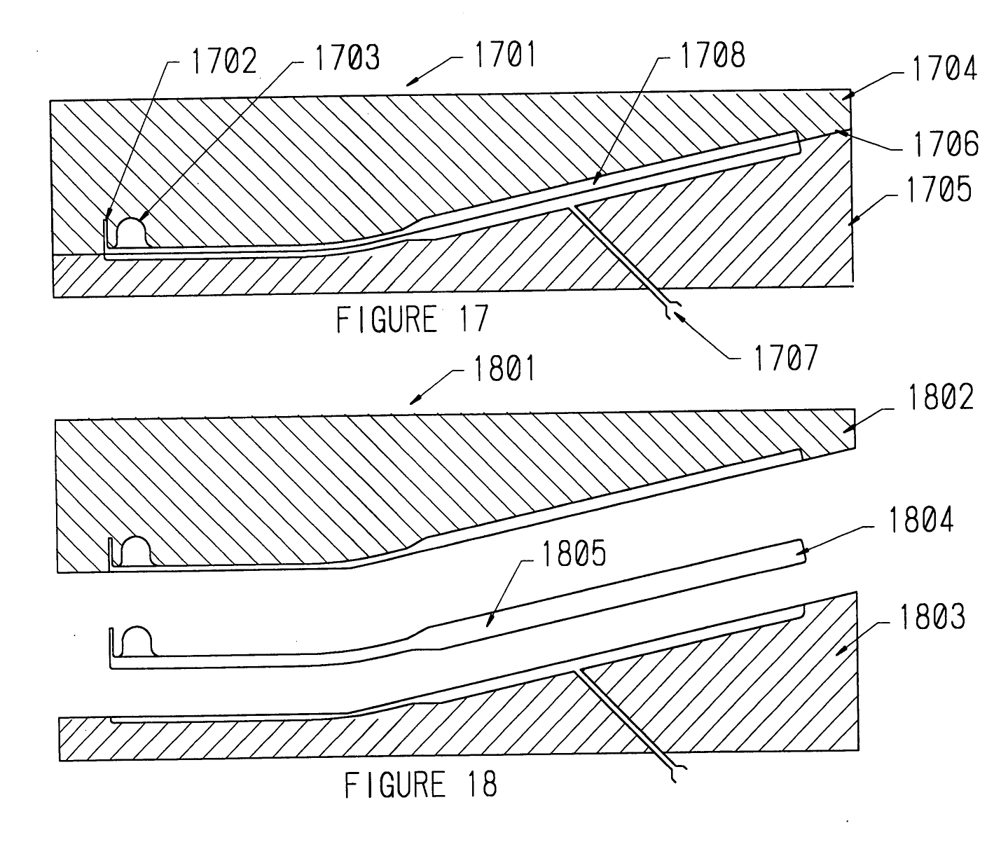

FIG. 16 depicts a longitudinal front view of one end of the movable drum roller guide 1601 as it is attached to the side of the device head 1602. The guide is built with a center indentation 1603 so as to fit over the rounded protrusion 1604 present in the head of the device 1602. This allows the drum roller to turn on the protrusion 1604 as the device is moved across the surface of the tongue. This method of attachment allows the drum roller guide and the tongue cleaning device head to be made separately and then snapped into place together after each has been manufactured.

The above material describes generally structures of some embodiments of the inventions. Below, a description is provided of equipment and apparatuses useful for injection molding various components of the tongue cleaners of the invention.

The general steps involved in injection molding a tongue cleaner of the invention include: (a) obtain or create a mold having an interior cavity reflective of the structure or tongue cleaner which is desired to be manufactured, (b) obtain a quantity of plastic or other suitable material for forming the tongue cleaner, (c) melt the plastic or other material until it is flowable, (d) force the flowable plastic or other material into the mold cavity, (e) permit the plastic or other material to cool and solidify in the cavity, (f) open the mold, and (g) remove the molded tongue cleaner. These steps will be elucidated in the text below in reference to the corresponding figures.

FIG. 17 shows a mold 1701 for injection molding a tongue cleaning device. This mold is used for a one-step unitary construction of a tongue cleaning device. This embodiment illustrates formation of a tongue cleaning device with a tongue scraper 1702 and a fixed guide 1703, although a tongue cleaning device with only a tongue scraper could be formed as desired. The mold 1701 includes a first mold half 1704 and a second mold half 1705. These two mold halves are separated by a centerline 1706. When the mold 1701 is fitted together as shown in FIG. 17, molding material is forced through the portal 1707 to form the tongue cleaner 1708. The formed tongue cleaner 1708 is then allowed to cool and solidify.

FIG. 18 shows an open mold 1801 following completion of injection molding from the previous figure. The top 1802 and bottom 1803 mold halves were separated along the centerline 1804 to yield a finished product tongue cleaner device 1805, which is of unitary construction.

FIG. 19 illustrates a tongue cleaning device 1901 formed via a multiple step injection molding process. This device 1901 is formed of two components each of which is individually molded. First, the tongue scraper 1902, cleaning device head 1904, and cleaning device handle 1903 were molded in the first stage. Then a second mold created the movable drum roller 1905 (see FIG. 21) was molded and then fitted into place in the device head 1904. The two separate components are affixed to each other mechanically by snapping the ends of the movable drum roller 1905 onto the attachment points 1906 formed as part of the cleaning device head 1904.

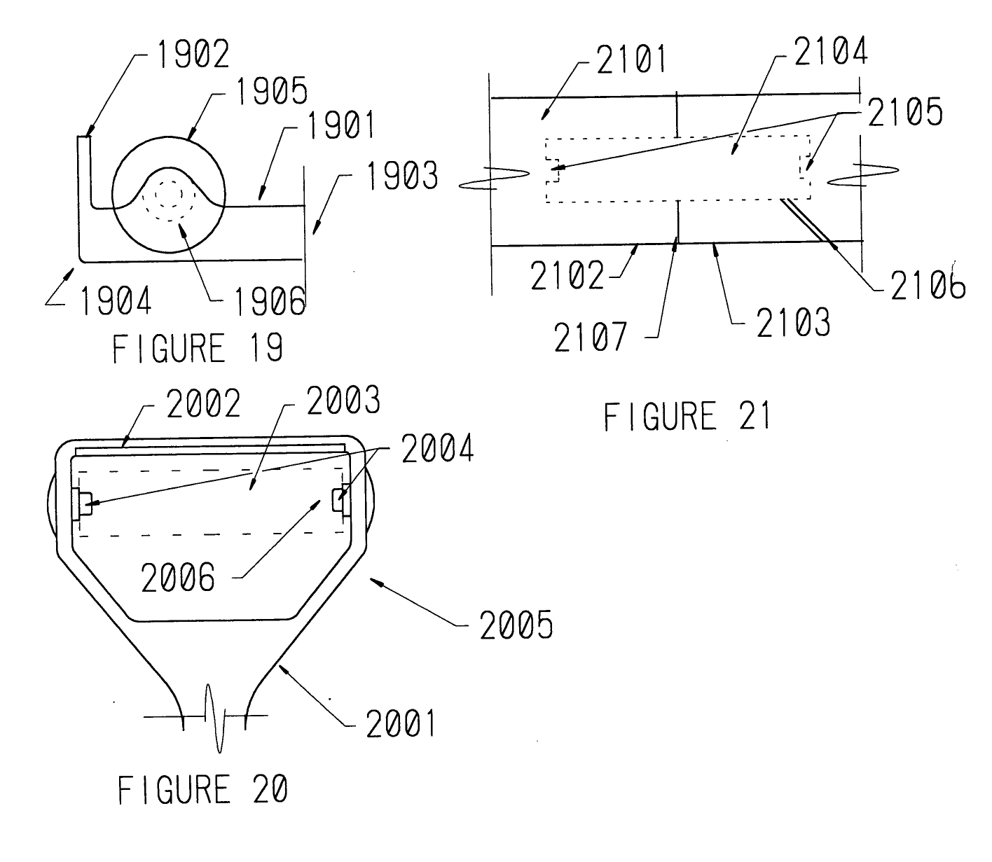

FIG. 20 shows a front view of the tongue cleaning device head 2001, which consists of the tongue scraper 2002 and the device head frame 2003, which surrounds an open space in the device head 2001. On either side of the space and as part of the frame 2003 two attachment points 2004 are formed as part of the frame 2003 in such a manner as to allow the indentations 2006 formed in the movable drum roller 2005 to be attached to the device head 2001. The attachment points 2004 are rounded and fit into the rounded indentations 2006 of the movable drum roller 2005 so that the movable drum roller will freely turn when the tongue cleaning device is pulled or pushed across the surface of the tongue.

FIG. 21 shows a mold 2101 for injection molding the movable drum roller. This mold is used for a one-step unitary construction of the movable drum roller. This embodiment illustrates formation of a movable drum roller 2104. The mold 2101 includes a first mold half 2102 and a second mold half 2103. These two mold halves are separated by a centerline 2107. When the mold 2101 is fitted together as shown in FIG. 21, molding material is forced through the portal 2106 to form the movable drum roller. After injection of the molding material the mold is allow to cool so the material will solidify. This mold pattern includes two indentations 2105, one on either end of the movable drum roller 2104 to facilitate the attachment of the movable drum roller to the head of the tongue cleaning device head (see FIGS. 19 and 20).

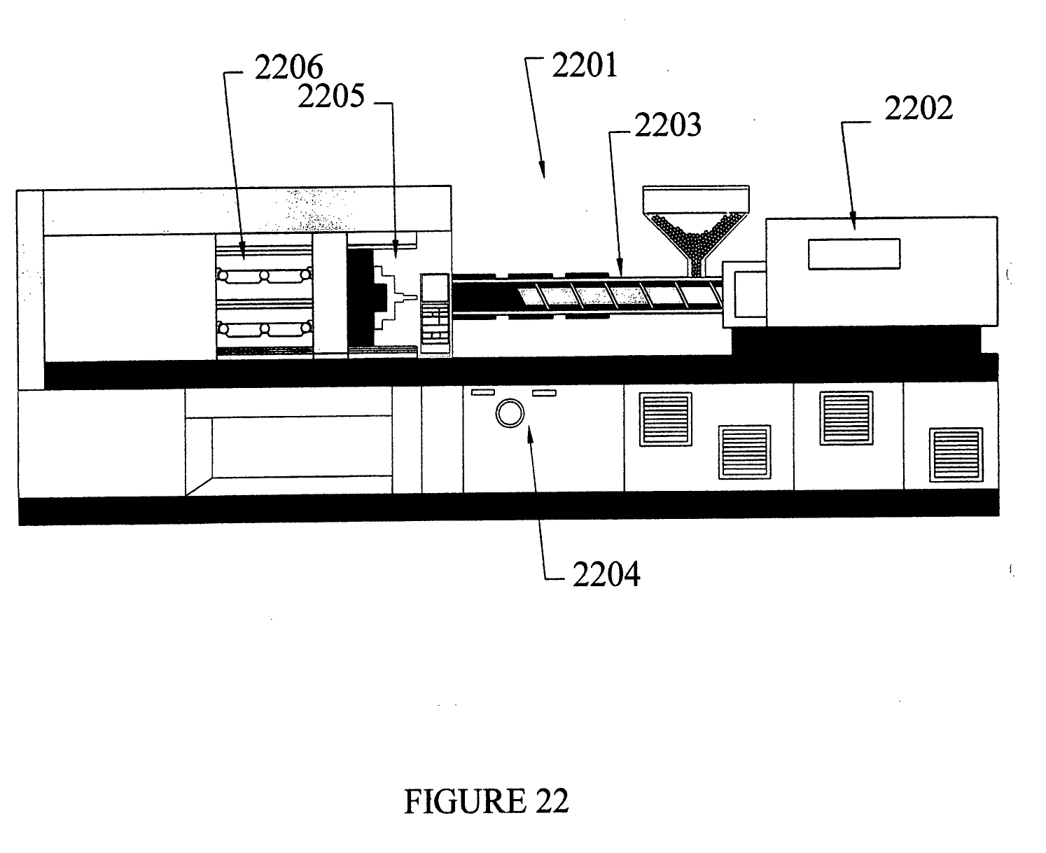

FIG. 22 illustrates an injection molding machine 2201 which may be used to make tongue cleaning device of the invention. The injection molding machine includes five systems. These systems include: the hydraulic system 2202, the injection system 2203, the control system 2204, the mold system 2205, and the clamping system 2206. The hydraulic system 2204 provides the power to run the injection system 2203, the mold system 2205, and the clamping system 2206. The injection system 2203 confines, melts and transports the plastic as it progresses through various stages in the machine 2201. The control system 2204 provides consistency and repeatability in machine operation. The mold system 2205 forms the molten plastic into a tongue cleaner. The clamping system 2206 opens and closes the mold, and supports and carries constituent parts of the mold.

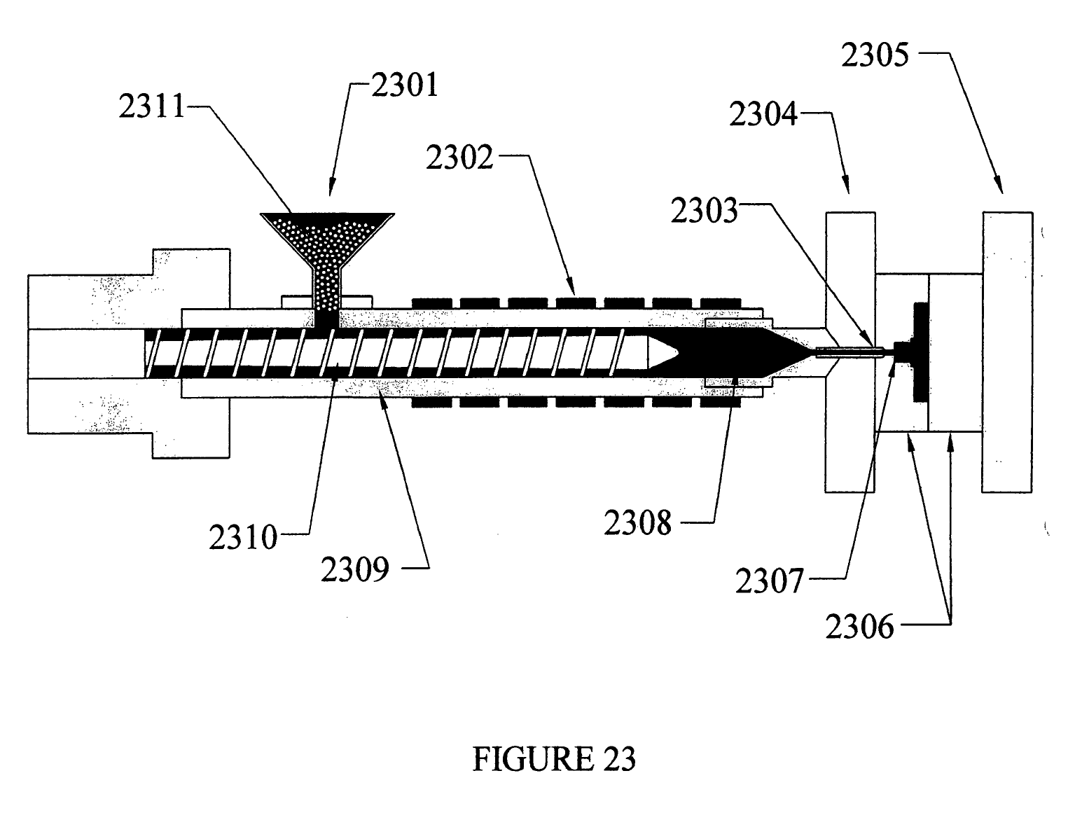

FIG. 23 illustrates the injection system of an injection molding machine useful when making injection molded tongue cleaners of the invention. Raw materials 2311, generally in the form of small pellets, are put into the hopper 2301. A barrel 2309 receives raw material 2311 from the hopper 2301 and supports a reciprocating plasticizing screw 2310. The barrel 2309 is heated by electric heater bands 2302, which melts the raw material that is located within the barrel 2309, permitting it to be plasticized by the turning screw 2310. The injection chamber 2308 delivers the material to the nozzle 2303. The nozzle 2303 connects the barrel 2309 and the mold 2306. Molten raw material from the barrel 2309 is forced by the screw 2310 through the nozzle 2303 into the sprue bushing 2307 and into the mold 2306 where it can assume the shape of the interior cavity of the mold. A movable platen 2305 detaches from a stationary platen 2304 to separate the mold halves and eject the molded tongue cleaner.

A discussion is provided below of some features and characteristics of some embodiments of the tongue cleaner inventions. This discussion should not be considered limited as to the scope of the inventions. A tongue cleaning device may consist of a head region, neck and handle. The head region is distally connected to the handle via the neck. The neck may be the narrowest portion of the tongue cleaning device. The handle is at the proximal end of the tongue cleaning device. The neck and/or handle may be angled to facilitate better angle of cleaning of the tongue by the tongue cleaning device.

One embodiment of the inventions consists of a tongue scraper and a fixed or rigid guide attached to the head region. The tongue cleaning device is approximately 32 mm long by 16-25 mm wide and 4 mm thick. The head with the scraper and guide are attached to the handle that is approximately 170 mm long, 12 mm wide and 5 mm thick. The handle can have finger grips formed of the same or a different material. The finger grips can be ridges or long strips of material. The grips are designed to facilitate handling and manipulating the tongue cleaning device.

The tongue cleaning device includes a scraper, which is approximately 4 mm high, 1 mm thick and 25 mm long and placed above the base of the head along the leading edge of the head. The fixed or rigid guide is placed in front of the scraper and runs parallel to the scraper from one side of the head to the other side. The top of the guide is a half circle in design being about 8 mm thick at the base and raising to a height of 4 mm and 25 mm long.

The surface of the rigid guide is smooth, however, variations of the surface may be used to permit the guide to drag along the tongue surface. One of the variations could be a series of random humps or bumps on the surface. Another variation could consist of a number of ridges running from one end of the guide to the other in parallel to each other. Another variation could be to construct a rough surface, similar to sand paper. All of these variations have the purpose of increasing the abrasive nature of the guide surface so as to aid in the cleaning of the tongue. The primary purpose of the guide, however, is to: (a) receive the primary pressure that is applied to the tongue cleaning device when the device is placed on the tongue surface (b) cause an indentation in the tongue surface in such a way as to present the tongue surface to the scraper at an angle rather than in a flat or parallel mode.

In an alternative embodiment of the tongue cleaning device the scraper is as described in paragraph 0055, however, the guide is composed of a movable drum roller designed to roll across the surface of the tongue rather than slide along the surface of the tongue. The drum roller causes an indentation in the tongue surface thus presenting the tongue surface to the scraper at an angle rather than in a flat or parallel mode. The drum roller also takes the primary pressure of the tongue cleaning devise and reduces the chance of the scraper causing any damage to the tender tongue surface tissue.

The surface of the movable drum roller guide is smooth, however, variation of the surface may be used to permit the guide to become involved in the cleaning process. One of the variations could be a series of random humps or bumps on the surface. Another variation could consist of a series of randomly placed bristles that are soft and pointed. The points of the bristles would penetrate the waste material that is held in the various crevices of the tongue surface, loosening them so that the scraper could carry the material to the front of the tongue to be discarded.

In another alternative embodiment of the tongue cleaning device the scraper is as described in paragraph 0052, and the guide is as described in paragraph 0056 and/or in paragraph 0057. In addition to the scraper and guide, this embodiment would include a tongue brush such as is described in FIG. 15. The tongue brush would consist of several individual bristles, pointed in nature, so as to be able to penetrate the small indentations of the tongue surface and loosen the waste material present in those crevices. The brush would loosen the waster material so that the scraper could carry that material to the front of the tongue to be discarded.

In some embodiments of the inventions, the tongue cleaning device may be made by injection molding. The first step is to obtain or create a suitable mold. The mold can be formed of machined metal such as aluminum or steel to have an interior cavity reflecting the shape of the tongue cleaner to be produced. The mold is then used in an injection molding machine which forces molten raw material, such as plastic, into the mold cavity to form the tongue cleaner. The injection molding process may be a single step or a series of steps, depending on the number of materials used. Possible materials for making the invention and components thereof include: polypropylene, polyethylene, polystyrene, thermoplastic rubber, resin, silicon rubber, natural rubber, plastic and other materials.

In one embodiment of the invention, the tongue cleaner may be formed by a one-step injection molding process wherein a single material, such as polypropylene, is used to form the entire tongue cleaning device such that a single mold is used to form all portions of the tongue cleaning device in a single step. The mold would yield the entire tongue cleaning device of unitary construction upon being opened.

An alternative embodiment is to use two or more molds wherein plastics, for example, of different degrees of rigidity would be molded separately to form distinct components of a tongue cleaning device. These separately molded portions can then be fitted together via a mechanical grip or by hand. One embodiment of a multiple step injection molding process is to have four molds, wherein the first mold forms the elongated member, the second forms the finger grips and scraper, the third mold forms the movable drum roller guide, and the fourth forms the tongue brush.

While the present invention has been described and illustrated in conjunction with a number of specific embodiments, those skilled in the art will appreciate that variations and modifications may be made without departing from the principles of the invention as herein illustrated, described, and claimed.

The present invention may be embodied in other specific forms without departing from its spirit or essential characteristics. The described embodiments are to be considered in all respects are only illustrative, and not restrictive. The scope of the invention is, therefore, indicated by the appended claims, rather than by the foregoing description. All changes, which come within the meaning and range of equivalency of the claims, are to be embraced within their scope.

1. A tongue cleaning device comprising:
a handle configured and adapted to be grasped by a human hand, the handle having a longitudinal axis,
a neck emanating from said handle,
having an orientation at an angle of less than 180 degrees with said handle longitudinal axis,
a head region located distal to said neck,
having a configuration to be substantially rigid platform that will support the scraper and scraper guide in use,
a tongue scraper located in said head region, said tongue scraper having a blade having sufficient flexibility to be useful in removing undesirable material from a human tongue by a scraping action,
said tongue scraper blade emanating from said tongue head base,
a fixed scraper guide located in said head region, said guide to be equal in height to the scraper and,
having a form of a half circle,
having a form of a half circle with the base of the guide attached to the head base, having a form of a half circle with the ridge running in parallel to the scraper, having a distance parallel to the scraper sufficient to allow the tongue surface to be indented by the guide but close enough to the scraper to allow the scraper to have contact with the tongue surface before it comes back to a normal position.

2. A device as recited in claim 1 wherein at least a portion of the tongue cleaning device includes a material selected from a group consisting of polypropylene, polyethylene, polystyrene, thermoplastic rubber, resin, silicon rubber, natural rubber and plastic. The device may be composed of a single material or a combination of two or more materials.

3. A device as recited in claim 1 further comprising finger grips located on said handle to enhance secure gripping of the handle.

4. A device as recited in claim 1
wherein the said scraper guide has a surface that is smooth.

5. A device as recited in claim 1
wherein the said scraper guide has a surface that has bumps or indentions which act to cause friction between the scraper guide and the tongue surface.

6. A device as recited in claim 1
wherein the said scraper guide has a surface that is rough like sandpaper which act to cause friction between the scraper guide and the tongue surface.

7. A device as recited in claim 1 wherein said handle, neck and head including scraper and scraper guide are formed as a single unitary component formed from a single material by an injection molding process.

8. A device as recited in claim 1
wherein the said scraper guide is a movable drum roller,
being a cylinder in design with an indentation on either end so as to be able to turn with the axis being the indentation on the end,
being a cylinder with indentations on either end so as to attach to the base of the tongue cleaning device in a way as to freely turn when the device is moved across the surface of the tongue,
being a cylinder of a diameter so that the height of the cylinder is equal in height to the height of the scraper,
being a cylinder of a length to extend the length of the device head and run parallel to the scraper,
being a cylinder attached to the head so that the distance between the cylinder and the scraper is such that the indentation made by the drum roller to the surface of the tongue will make contact with the scraper before the tongue surface comes back to normal configuration.

9. A device as recited in claim 8
wherein the surface of the movable drum roller is smooth.

10. A device as recited in claim 8
wherein the surface of the movable drum roller has bumps or indentations randomly placed on the entire surface of the drum roller.

11. A device as recited in claim 8
wherein the surface of the movable drum roller has a series of individual pointed soft pliable bristles randomly placed on the entire drum roller surface of sufficient length and strength to reach into the indentations of the tongue surface yet soft enough to not injure the tender soft tongue tissue.

12. A device as recited in claim 8 wherein said handle, neck, head, scraper and movable drum roller guide are formed from materials selected from the group of materials consisting of polypropylene, polyethylene, polystyrene, thermoplastic rubber, resin, silicon rubber, natural rubber and plastic.

13. A device as recited in claim 8
wherein said handle, neck and head are from in one injection molding process of a certain material and the movable drum roller guide is formed in a second injection molding process of a certain material and then snapped into place by a mechanical guide or by hand; and
wherein the first material and said second material may or may not differ from each other.

14. A device as recited in claim 1
wherein a tongue brush is added to the head just in front of the scraper guide, having a form of a series of single brush bristles,
the bristles being pointed in nature rising from the head base outwardly,
the bristles having a height equal to the top of the scraper guide and scraper, the bristles being soft and pliable,
the bristles have a base that is not more than about 1 mm in diameter,
the bristles are of a length greater than about 5 mm and less than about 10 mm.

15. A device as cited in claim 14
wherein a plurality of bristles emanating from said bristle base, each of said bristles having a proximal end adjacent said bristle base and a distal end, at least some of said bristles being tapered from their proximal end to their distal end to presented a pointed bristle appearance,
said bristles being flexible to accommodate brushing of a human tongue without causing injury to the tongue,
said bristles and said bristle base being formed as a single unitary component from the same material via an injection molding process, and
said bristles emanating from said bristle base in an individually-mounted and free-standing non-tufted configuration.

16. A device as cited in claim 14
wherein said bristles are arranged on said bristle base in a row or in a colony formation.

17. A device as recited in claim 14
wherein said handle, neck, head, scraper and guide are formed in one injection molding process of a certain material,
wherein said tongue brush is formed in a second injection molding process of a certain material,
wherein said handle, neck, head, scraper and guide are combined with said tongue brush by a mechanical or hand process.

18. A device as recited in claim 8
where a tongue brush is added to the head just in front of the movable drum roller, having a form of a series of single brush bristles,
the bristles being pointed in nature rising from the head base outwardly,
the bristles having a height equal to the top of the scraper guide and movable drum roller,
the bristles being soft and pliable.

19. A device as cited in claim 18
wherein a plurality of bristles emanating from said bristle base, each of said bristles having a proximal end adjacent said bristle base and a distal end, at least some of said bristles being tapered from their proximal end to their distal end to presented a pointed bristle appearance,
said bristles being flexible to accommodate brushing of a human tongue without causing injury to the tongue,
said bristles and said bristle base being formed as a single unitary component from the same material via an injection molding process, and
said bristles emanating from said bristle base in an individually-mounted and free-standing non-tufted configuration.

20. A device as cited in claim 18
wherein said bristles are arranged on said bristle base in a row or in a colony formation.

21. A device as recited in claim 18
wherein said handle, neck, head, and scraper are formed in one injection molding process of a certain material,
wherein said movable drum roller is formed in a second injection molding process of a certain material,
wherein said tongue brush is formed in a third injection molding process of a certain material,
wherein said components of the tongue cleaning device is combined together either in a mechanical or hand process.
